(19) United States
(12) Patent Application Publication
(10) Pub. No.: US 2022/0410160 A1
(43) Pub. Date: Dec. 29, 2022

(54) MICROFLUIDIC CHIP AND MICROFLUIDIC DEVICE

(71) Applicants: BEIJING BOE TECHNOLOGY DEVELOPMENT CO., LTD., Beijing (CN); BOE TECHNOLOGY GROUP CO., LTD., Beijing (CN)

(72) Inventors: Houqian GAO, Beijing (CN); Beiyuan FAN, Beijing (CN); Ding DING, Beijing (CN)

(21) Appl. No.: 17/780,194

(22) PCT Filed: Jun. 9, 2021
(86) PCT No.: PCT/CN2021/099085

§ 371 Date: May 26, 2022

(51) Int. Cl. B01L 3/00

(52) U.S. Cl. CPC B01L 3/502761; B01L 2200/0663; B01L 2300/0851; B01L 2300/0864; B01L 2300/0893

(57) ABSTRACT

The present disclosure provides a microfluidic chip including a plurality of microcavities, at least two of the plurality of microcavities have different volumes.

### TECHNICAL FIELD

The present disclosure relates to the field of microfluidics, and in particular, to a microfluidic chip and a microfluidic device comprising the microfluidic chip.

### BACKGROUND

Polymerase Chain Reaction (PCR) is a molecular biology technique used to amplify specific DNA fragments, which is able to replicate tiny amounts of DNA in large numbers, greatly increasing its numbers. During the PCR reaction, the double-chain structure of the DNA fragment is denatured at high temperature (e.g., 95° C.) to form a single-chain structure. The primer and the single chain are combined at low temperature (e.g., 60° C.) according to the principle of base complementary pairing. At the optimal temperature (e.g., 70° C.) of DNA polymerase, the base-binding extension is achieved, and DNA polymerase synthesizes complementary chains along the direction of phosphate to five-carbon sugar (5′-3′). The above process is the temperature cycle process of denaturation-annealing-extension. Through multiple temperature cycle processes of denaturation-annealing-extension, DNA fragments can achieve large-scale replication. Digital polymerase chain reaction (dPCR) technology is a quantitative analysis technology developed on the basis of PCR that can provide quantitative information of digital DNA, which can further improve the sensitivity and accuracy of detection, so it has attracted more and more attention.

### SUMMARY

According to an aspect of the present disclosure, a microfluidic chip is provided. The microfluidic chip comprises a plurality of microcavities, at least two of the plurality of microcavities have different volumes.

In some embodiments, the plurality of microcavities comprise at least three types of microcavities with different volumes, and a ratio of volumes of the at least three types of microcavities with different volumes is 1:2˜4:3˜8.

In some embodiments, the ratio of volumes of the at least three types of microcavities with different volumes is 1:4:8.

In some embodiments, the plurality of microcavities comprise at least one first microcavity, at least one second microcavity and at least one third microcavity, the first microcavity, the second microcavity and the third microcavity have a same depth, a ratio of an area of a bottom of the first microcavity, an area of a bottom of the second microcavity, and an area of a bottom of the third microcavity is 1:4:8.

In some embodiments, a shape of an orthographic projection of the bottom of the first microcavity, a shape of an orthographic projection of the bottom of the second microcavity and a shape of an orthographic projection of the bottom of the third microcavity on the microfluidic chip are circular.

In some embodiments, a radius of the bottom of the first microcavity is 20˜30 μm, a radius of the bottom of the second microcavity is 40˜60 μm, and a radius of the bottom of the third microcavity is 56.57˜84.85 μm.

In some embodiments, the depths of the first microcavity, the second microcavity and the third microcavity are 30˜70 μm.

In some embodiments, the first microcavities, the second microcavities and the third microcavities are arranged in an array, in a first direction, a row of second microcavities is arranged between two adjacent rows of third microcavities, and in a second direction, a column of second microcavities is arranged between two adjacent columns of third microcavities.

In some embodiments, a distance between the centers of the bottoms of two adjacent first microcavities in the first direction is equal to a distance between the centers of the bottoms of two adjacent first microcavities in the second direction; a distance between the centers of the bottoms of two adjacent second microcavities in the first direction is equal to a distance between the centers of the bottoms of two adjacent second microcavities in the second direction; and a distance between the centers of the bottoms of two adjacent third microcavities in the first direction is equal to a distance between the centers of the bottoms of two adjacent third microcavities in the second direction.

In some embodiments, an intersection of the third microcavities in two adjacent rows and the third microcavities in two adjacent columns comprises four third microcavities, lines connecting centers of the bottoms of the four third microcavities form a square, one second microcavity is arranged at a center of the four third microcavities, and a center of the bottom of the second microcavity coincides with a midpoint of a diagonal of the square; in the first direction or the second direction, one first microcavity is arranged between any two adjacent third microcavities, a center of the bottom of the first microcavity coincides with a midpoint of the line connecting centers of the bottoms of the two adjacent third microcavities; and in the first direction or the second direction, one first microcavity is arranged between any two adjacent second microcavities, a center of the bottom of the first microcavity coincides with a midpoint of the line connecting centers of the bottoms of the two adjacent second microcavities.

In some embodiments, an area of an orthographic projection of the plurality of microcavities on the microfluidic chip accounts for 76.82% of an area of the microfluidic chip.

In some embodiments, the ratio of volumes of the at least three types of microcavities with different volumes is 1:2:3.

In some embodiments, the plurality of microcavities comprise at least one first microcavity, at least one second microcavity and at least one third microcavity, the first microcavity, the second microcavity and the third microcavity have a same depth. A shape of an orthographic projection of the first microcavity on the microfluidic chip is an equilateral triangle, a shape of an orthographic projection of the second microcavity on the microfluidic chip is a parallelogram, a shape of an orthographic projection of the third microcavity on the microfluidic chip is a trapezoid, and a ratio of an area of the equilateral triangle, an area of the parallelogram, and an area of the trapezoid is 1:2:3.

In some embodiments, the first microcavities, the second microcavities and the third microcavities are arranged in an array, in a first direction, each row is alternately arranged in a sequence of the first microcavity, the second microcavity and the third microcavity, and in a second direction, the microcavities in a same column have a same shape.

In some embodiments, in each row, a first side of the parallelogram and a first side of the equilateral triangle adjacent to the parallelogram are parallel to each other, and a distance between the first side of the parallelogram and the first side of the equilateral triangle adjacent to the parallelogram is a first distance; a second side of the parallelogram and a first side of the trapezoid adjacent to the parallelogram are parallel to each other, and a distance between the second side of the parallelogram and the first side of the trapezoid adjacent to the parallelogram is a second distance; a second side of the adjacent trapezoid and a second side of the equilateral triangle adjacent to the adjacent trapezoid are parallel to each other, and a distance between the second side of the adjacent trapezoid and the second side of the equilateral triangle adjacent to the adjacent trapezoid is a third distance. The first distance, the second distance and the third distance are the same.

In some embodiments, a length of each of four sides of the parallelogram is equal to a length of a side of the equilateral triangle, a length of an upper base of the trapezoid is equal to the length of the side of the equilateral triangle, a length of a lower base of the trapezoid is twice the length of the side of the equilateral triangle, the parallelogram consists of two equilateral triangles, and the trapezoid consists of three equilateral triangles.

In some embodiments, the depths of the first microcavity, the second microcavity and the third microcavity are 30˜70 μm.

In some embodiments, an area of an orthographic projection of the plurality of microcavities on the microfluidic chip accounts for 72.90% of an area of the microfluidic chip.

In some embodiments, the ratio of volumes of the at least three types of microcavities with different volumes is 1:2:4.

In some embodiments, the plurality of microcavities comprise at least one first microcavity, at least one second microcavity and at least one third microcavity, an area of a bottom of the first microcavity, an area of a bottom of the second microcavity and an area of a bottom of the third microcavity are the same.

In some embodiments, a depth of the first microcavity is 25˜40 μm, a depth of the second microcavity is 50˜80 μm, and a depth of the third microcavity is 100˜160 μm.

In some embodiments, shapes of an orthographic projection of the bottom of the first microcavity, an orthographic projection of the bottom of the second microcavity and an orthographic projection of the bottom of the third microcavity on the microfluidic chip are circular, and a radius of the bottom of the first microcavity, a radius of the bottom of the second microcavity, and a radius of the bottom of the third microcavity are the same.

In some embodiments, the plurality of microcavities are arranged in a manner of two-dimensional hexagonal dense arrangement or two-dimensional square lattice, and a spacing between any two adjacent microcavities of the plurality of microcavities is 10˜80 μm.

In some embodiments, an area of an orthographic projection of the plurality of microcavities on the microfluidic chip accounts for 24.67%˜68.43% of an area of the microfluidic chip.

In some embodiments, the plurality of microcavities are arranged in the manner of two-dimensional hexagonal dense arrangement, and the spacing between any two adjacent microcavities of the plurality of microcavities is 50 μm, and the area of the orthographic projection of the plurality of microcavities on the microfluidic chip accounts for 40.18% of the area of the microfluidic chip.

According to another aspect of the present disclosure, a microfluidic device is provided. The microfluidic device comprises the microfluidic chip described in any one of the preceding embodiments.

### BRIEF DESCRIPTION OF THE DRAWINGS

In order to describe the technical solutions in the embodiments of the present disclosure more clearly, the accompanying drawings required in the embodiments will be briefly introduced below. Obviously, the drawings in the following are only some embodiments of the present disclosure. For those of ordinary skill in the art, other drawings can also be obtained from these drawings without any creative effort.

FIG. 1 illustrates a schematic diagram of a base substrate of a microfluidic chip according to an embodiment of the present disclosure;

FIG. 2 illustrates a schematic structural diagram of a microfluidic chip according to an embodiment of the present disclosure;

FIG. 3 illustrates an arrangement of the microcavities of the microfluidic chip according to an embodiment of the present disclosure;

FIG. 4 illustrates a cross-sectional view taken along the line AA′ in FIG. 3;

FIG. 5 illustrates a cross-sectional view taken along the line BB′ in FIG. 3;

FIG. 6 illustrates an arrangement of the microcavities of a microfluidic chip according to another embodiment of the present disclosure;

FIG. 7 illustrates a cross-sectional view taken along the line CC′ in FIG. 6;

FIG. 8 illustrates an arrangement of the microcavities of a microfluidic chip according to yet another embodiment of the present disclosure;

FIG. 9 illustrates an arrangement of the microcavities of the microfluidic chip of FIG. 8;

FIG. 10 illustrates another arrangement of the microcavities of the microfluidic chip of FIG. 8;

FIG. 11 illustrates a cross-sectional view taken along the line DD′ in FIG. 10; and

FIG. 12 illustrates a block diagram of a microfluidic device according to yet another embodiment of the present disclosure.

### DETAILED DESCRIPTION OF THE DISCLOSURE

The technical solutions in the embodiments of the present disclosure will be clearly and completely described below with reference to the accompanying drawings in the embodiments of the present disclosure. Obviously, the described embodiments are only some, but not all, embodiments of the present disclosure. Based on the embodiments in the present disclosure, all other embodiments obtained by those of ordinary skill in the art without creative efforts shall fall within the protection scope of the present disclosure.

DPCR has been widely used in fields such as clinical diagnosis, gene instability analysis, single-cell gene expression, environmental microbial detection and prenatal diagnosis due to its advantages of high sensitivity, strong specificity, high detection throughput, and accurate quantification. dPCR technology is an absolute quantification technology of nucleic acid molecules, the principle of which can be roughly described as: fully dilute the sample solution comprising the target nucleic acid molecules (the target nucleic acid molecules that this application hopes to study, such as nucleic acid molecules of cancer cells), then distribute the diluted sample solution into a large number of tiny reaction cells of the microfluidic chip, so that each reaction cell comprises one or zero nucleic acid molecule. PCR amplification of single molecule is then performed in each reaction cell to form a solution to be detected. Then a fluorescence microscope or flow cytometer is used to detect the fluorescence intensity of the solution to be tested in each reaction cell, and finally calculate the number (or concentration) of target nucleic acid molecules in the original sample through the number of positive reaction cells and Poisson distribution statistics, so as to achieve absolute quantification.

In the dPCR reaction, the number of nucleic acid molecules in the diluted sample solution is usually less than the number of reaction cells of the microfluidic chip, so that the distribution of the number of nucleic acid molecules in each reaction cell satisfies the Poisson distribution probability model. According to the Poisson distribution formula, it can be concluded that: N=−1n(1−P), where N represents the number of nucleic acid molecules contained in each reaction cell, and P represents the proportion of negative cells in all reaction cells. The upper limit of quantification of dPCR mainly depends on the volume and number of reaction cells, while the lower limit of detection is related to the total volume of the sample solution.

The term “dynamic range” refers to linear dynamic range. Specifically, it means that within a certain concentration range, the known concentration of the sample and the concentration of the sample obtained by measurement exhibit an acceptable linear relationship. The unit of dynamic range is usually expressed in logs. For example, the sample solution is serially diluted for many times, such as 5 serial dilutions of 10 times, a total of 6 serially diluted sample solutions (100000X, 10000X, 1000X, 100X, 10X, 1X) can be obtained, then the 6 serially diluted sample solutions can be detected to obtain their detection concentrations (such as mmol/mL) respectively, then calculate the linearity between the concentrations of the sample solutions of the six known dilution ratios and their respective detection concentrations. If the linearity reaches the predetermined threshold, it means that the detection can reach a dynamic range of 6 logs.

The inventors of the present application found that, in the conventional technology, whether it is droplet dPCR or microwell dPCR, the volumes of multiple reaction cells of the microfluidic chip are consistent. Since the volume of each reaction cell is the same, the number of nucleic acid molecules contained in the sample solution accommodated in each reaction cell is basically the same in theory. In order to comprise at most one nucleic acid molecule in each reaction cell, it is therefore necessary to dilute the sample solution to a fixed concentration (i.e., a fixed multiple). However, the dynamic range and sensitivity of dPCR cannot be adjusted in this way, which greatly reduces the experimental efficiency. Moreover, in the case of reaction cell with such a single-volume, in order to make the sample concentration fall into the concentration range suitable for dPCR, it is usually necessary to perform multiple serial dilutions of the sample, but this serial dilution method increases the amount of related reagents and brings the risk of cross-contamination (e.g., with contaminants in the environment).

In view of this, embodiments of the present disclosure provide a microfluidic chip comprising a plurality of microcavities, and at least two of the plurality of microcavities have different volumes.

The microfluidic chip provided by the embodiments of the present disclosure can achieve multiple dynamic ranges, because the microfluidic chip can allow a larger selection range for the dilution concentration of the sample solution, rather than only diluting to a fixed concentration. When the diluted sample solution is applied to the microfluidic chip, since the microcavities of the microfluidic chip comprise microcavities with a large volume and microcavities with a small volume, the microcavity with the large volume should comprise more nucleic acid molecules in the sample solution than the microcavity with the small volume. Therefore, if the concentration of the diluted sample solution is low, since the microfluidic chip comprises microcavities with the large volume, the solution contained in the microcavity with the large volume can satisfy the requirement of comprising one nucleic acid molecule in the microcavity (the number of nucleic acid molecules in the microcavity with the large volume=concentration of the diluted sample solution (mmol/mL*microcavity volume (mL)). If the concentration of the diluted sample solution is high, the number of nucleic acid molecules contained in the microcavity with the large volume may exceed the threshold requirement. However, since the microfluidic chip also includes microcavities with the small volume, the solution contained in the microcavity with the small volume can satisfy the requirement of comprising one nucleic acid molecule in the microcavity (the number of nucleic acid molecules in the microcavity with the small volume=the concentration of the diluted sample solution (mmol/mL* microcavity volume (mL)). Therefore, whether the concentration of the diluted sample solution is slightly higher or lower, the microfluidic chip can be adapted, so that multiple dynamic ranges can be achieved without only diluting the sample solution to a fixed factor as in the related art.

For example, if a solution with a concentration of C1 is added to each microcavity, the concentration of C1 is sufficient for a microcavity with the large volume to comprise one nucleic acid molecule, so the initial number of molecules of target nucleic acid molecules in the sample solution can be calculated mainly according to the measured value of the sample solution in the microcavity with the large volume. If a solution with a concentration of C2 (C2>C1) is added to each microcavity, the concentration of C2 may be too high for the microcavity with the large volume, making the number of nucleic acid molecules of the sample solution in each microcavity with the large volume exceed the threshold requirement. However, the concentration of C2 may be sufficient for a microcavity with the small volume to comprise one nucleic acid molecule, so the initial number of molecules of the target nucleic acid molecules in the sample solution can be calculated mainly according to the measured value of the sample solution in the microcavity with the small volume. The microcavity with the small volume is essentially equivalent to a greater dilution of the sample solution relative to the microcavity with the large volume, so it can adapt to higher concentration solutions. In this way, it is possible to allow the sample solution to be diluted over a larger concentration range (for example, it can choose to dilute to concentration C1 or C2), rather than having to be diluted to a fixed multiple as in the related art. Therefore, compared with the microfluidic chip in the related art, the microfluidic chip provided by the embodiments of the present disclosure realizes the expansion of the dynamic range, improves the detection sensitivity, and simultaneously realizes multiple detection lines on a single microfluidic chip, thus improving the experimental efficiency. In addition, compared with the conventional technology that requires multiple serial dilutions of samples to meet the concentration requirements of a microcavity with the single volume, the microfluidic chip provided by the embodiments of the present disclosure avoids multiple serial dilutions of the sample, thereby avoiding the waste of reagents and the risk of cross-contamination.

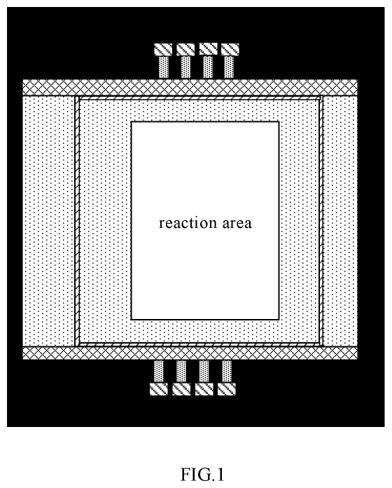

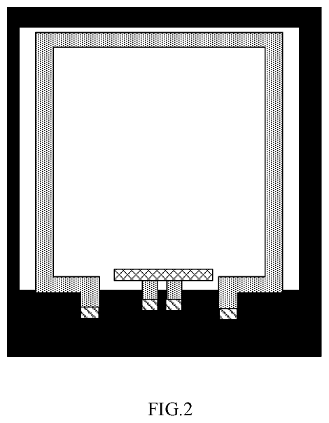

A microfluidic chip usually comprises an upper cover integrated with a gas valve structure, a base substrate, a temperature control module, a program-controlled voltage unit, a sample reagent mixed liquid inlet and outlet, and other structures. FIG. 1 illustrates a schematic diagram of the base substrate of the microfluidic chip, and FIG. 2 illustrates a schematic diagram of the microfluidic chip after the upper cover and the base substrate are assembled. As illustrated in the figure, a reaction area is arranged in the center of the base substrate, and a plurality of microcavities as described above are arranged in the reaction area. The plurality of microcavities comprise at least three types of microcavities with different volumes, and the ratio of volumes of the at least three types of microcavities with different volumes may be 1:2˜4:3˜8.

The arrangement of the microcavities with different volumes of the microfluidic chip will be described below with several specific embodiments.

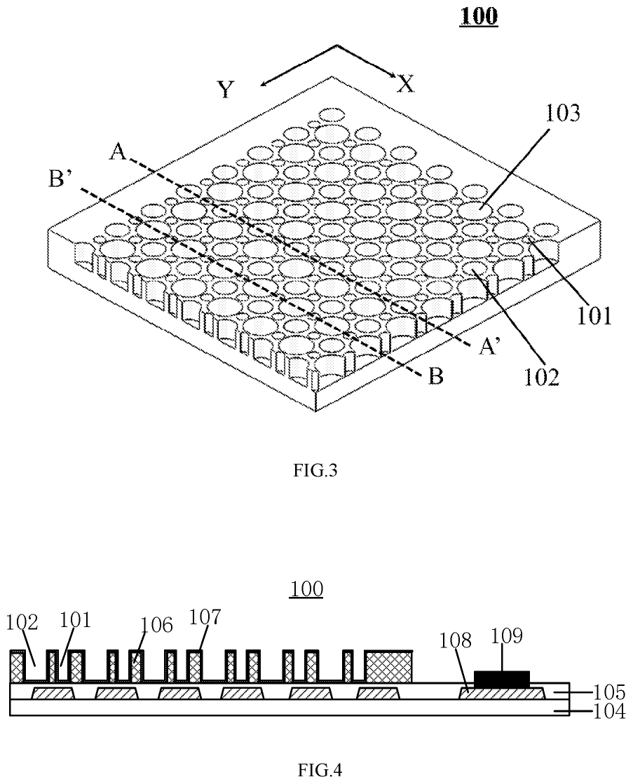

FIG. 3 illustrates a partial top view of the microfluidic chip 100, FIG. 4 illustrates a cross-sectional view taken along line AA′ in FIG. 3, and FIG. 5 illustrates a cross-sectional view taken along line BB′ in FIG. 3.

As illustrated in FIGS. 3-5, the microfluidic chip 100 comprises a plurality of microcavities comprising at least one first microcavity 101, at least one second microcavity 102 and at least one third microcavity 103. The first microcavity 101, the second microcavity 102 and the third microcavity 103 are all cylindrical and have the same depth, for example, the depths of the first microcavity 101, the second microcavity 102 and the third microcavity 103 are all 30˜70 μm. In an example, the depths of the first microcavity 101, the second microcavity 102 and the third microcavity 103 are all 50 μm. The volume of the first microcavity 101: the volume of the second microcavity 102: the volume of the third microcavity 103 is less than or equal to 1:4:8. In an example, the volume of the first microcavity 101: the volume of the second microcavity 102: the volume of the third microcavity 103 is equal to 1:4:8. In another example, the volume of the first microcavity 101: the volume of the second microcavity 102: the volume of the third microcavity 103 is equal to 1:4:6. In yet another example, the volume of the first microcavity 101: the volume of the second microcavity 102: the volume of the third microcavity 103 is equal to 1:3:5. The embodiments of the present disclosure do not specifically limit the specific ratio of the volumes of the first microcavity 101, the second microcavity 102 and the third microcavity 103, as long as the ratio is less than or equal to 1:4:8.

Since the first microcavity 101, the second microcavity 102 and the third microcavity 103 are all cylindrical, the bottoms of the first microcavity 101, the second microcavity 102 and the third microcavity 103 are all circular. The radius of the bottom of the first microcavity is 20˜30 μm, the radius of the bottom of the second microcavity is 40˜60 μm, and the radius of the bottom of the third microcavity is 56.57˜84.85 μm. In an example, the radius of the bottom of the first microcavity 101 is 25 μm, the radius of the bottom of the second microcavity 102 is 50 μm, and the radius of the bottom of the third microcavity 103 is 70.71 μm. The area of the bottom of the first microcavity 101: the area of the bottom of the second microcavity 102: the area of the bottom of the third microcavity 103 is equal to 1:4:8.

As shown in the figure, the first microcavities 101, the second microcavities 102 and the third microcavities 103 are all arranged in an array. In the first direction X, a row of second microcavities 102 is arranged between two adjacent rows of third microcavities 103, in the second direction Y, a column of second microcavities 102 is arranged between two adjacent columns of third microcavities 103, and in the first direction X or the second direction Y, one first microcavity 101 is arranged between any two adjacent second microcavities 102, and one first microcavity 101 is arranged between any two adjacent third microcavities 103. The distance between the centers of the bottoms of two adjacent first microcavities 101 in the first direction X is equal to the distance between the centers of the bottoms of two adjacent first microcavities 101 in the second direction Y; the distance between the centers of the bottoms of two adjacent second microcavities 102 in the first direction X is equal to the distance between the centers of the bottoms of two adjacent second microcavities 102 in the second direction Y; the distance between the centers of the bottoms of the two adjacent third microcavities 103 in the first direction X is equal to the distance between the centers of the bottoms of the two adjacent third microcavities 103 in the second direction Y. In an example, in the first direction X, the distance between the centers of the bottoms of two adjacent first microcavities 101 is 200 μm, and in the second direction Y, the distance between the centers of the bottoms of two adjacent first microcavities 101 is 200 μm. In the first direction X, the distance between the centers of the bottoms of two adjacent second microcavities 102 is 200 μm, and in the second direction Y, the distance between the centers of the bottoms of two adjacent second microcavities 102 is 200 μm. In the first direction X, the distance between the centers of the bottoms of two adjacent third microcavities 103 is 200 μm, and in the second direction Y, the distance between the centers of the bottoms of two adjacent third microcavities 103 is 200 μm. That is, the intersection of the N^{th} row and the (N+2)^{th} row (N≥1) of the first microcavities 101 and two adjacent columns of the first microcavities 101 comprises four first microcavities 101. The lines connecting the centers of the bottoms of the four first microcavities 101 form a square with a side length of 200 μm. The intersection of two adjacent rows of second microcavities 102 and two adjacent columns of second microcavities 102 comprises four second microcavities 102. The lines connecting the centers of the bottoms of the four second microcavities 102 form a square with a side length of 200 μm. The intersection of two adjacent rows of third microcavities 103 and two adjacent columns of third microcavities 103 comprises four third microcavities 103. The lines connecting centers of the bottoms of the four third microcavities 103 form a square with a side length of 200 μm.

As illustrated in the figure, the intersection of two adjacent rows of third microcavities 103 and two adjacent columns of third microcavities 103 comprises four third microcavities 103, and the lines connecting the centers of the bottoms of the four third microcavities 103 form a square (e.g., the side length of the square is 200 μm), one second microcavity 102 is arranged in the center of the four third microcavities 103, and the center of the bottom of the second microcavity 102 coincides with the midpoint of the diagonal of the square. In the first direction X or the second direction Y, one first microcavity 101 is arranged between any two adjacent third microcavities 103, and the center of the bottom of the first microcavity 101 coincides with the midpoint of the line connecting the centers of the bottoms of the two adjacent third microcavities 103. In the first direction X or the second direction Y, one first microcavity 101 is arranged between any two adjacent second microcavities 102, and the center of the bottom of the first microcavity 101 coincides with the midpoint of the line connecting the centers of the bottoms of the two adjacent second microcavities 102. Therefore, it can be obtained through calculation that the distance between the center of the bottom of any third microcavity 103 in the four third microcavities 103 enclosed in a square and the center of the bottom of the second microcavity 102 arranged in the center of the square is 141.4 μm, the distance between the center of the bottom of the third microcavity 103 and the center of the bottom of the first microcavity 101 adjacent thereto is 100 μm, and the distance between the center of the bottom of the second microcavity 102 and the center of the bottom of the first microcavity 101 adjacent thereto is 100 μm. Therefore, the third microcavity 103 and the first microcavity 101 are spaced apart from each other, the third microcavity 103 and the second microcavity 102 are spaced apart from each other, and the first microcavity 101 and the second microcavity 102 are spaced apart from each other. Such distribution manner can prevent the mutual interference among different microcavities, and is also favorable to the effective identification of each microcavity by fluorescence microscopy.

In some embodiments, the area of the orthographic projection of the plurality of microcavities on the microfluidic chip 100 accounts for 76.82% of the area of the microfluidic chip 100.

It should be noted that although in FIG. 3, the first microcavity 101, the second microcavity 102 and the third microcavity 103 are all cylindrical, this is only an example, and the embodiment of the present disclosure does not limit the specific shapes of the first microcavity 101, the second microcavity 102, and the third microcavity 103. For example, the shapes of the first microcavity 101, the second microcavity 102 and the third microcavity 103 comprise, but are not limited to, a cube, a quadrangular prism, a regular polyhedron, and the like.

The microfluidic chip 100 has three microcavities with different volumes, that is, the ratio of the volumes of the first microcavity 101, the second microcavity 102 and the third microcavity 103 with the same depth but different bottom areas is 1:4:8. The microfluidic chip 100 can allow the dilution concentration of the sample solution to have a larger selection range, without being restricted to only be diluted to a fixed concentration. For example, if a solution with a concentration of C1 is added to each microcavity, the concentration of C1 is sufficient for the third microcavity 103 to comprise one nucleic acid molecule, so the initial number of molecules of target nucleic acid molecules in the sample solution can be calculated mainly according to the measured value of the sample solution in the third microcavity 103. If a solution with a concentration of C2

(C2>C1) is added to each microcavity, the concentration of C2 may be too high for the third microcavity 103, making the number of nucleic acid molecules of the sample solution in each third microcavity 103 exceed the threshold requirement. However, the concentration of C2 may be sufficient for the second microcavity 102 to comprise one nucleic acid molecule, so the initial number of molecules of the target nucleic acid molecules in the sample solution can be calculated mainly according to the measured value of the sample solution in the second microcavity 102. If a solution with a concentration of C3 (C3>C2>C1) is added to each microcavity, the concentration of C3 may be too high for the third microcavity 103 and the second microcavity 102, making the number of nucleic acid molecules of the sample solution in each third microcavity 103 and each second microcavity 102 exceed the threshold requirement. However, the concentration of C3 may be sufficient for the first microcavity 101 to comprise one nucleic acid molecule, so the initial number of molecules of the target nucleic acid molecules in the sample solution can be calculated mainly according to the measured value of the sample solution in the first microcavity 101. The microcavity with the smaller volume is essentially equivalent to a greater dilution of the sample solution relative to the microcavity with the larger volume, so it can adapt to higher concentration solutions. In this way, it is possible to allow the sample solution to be diluted over a larger concentration range (for example, it can choose to dilute to concentration C1, C2 or C3), rather than having to be diluted to a fixed multiple as in the related art. Therefore, compared with the microfluidic chip in the related art, the microfluidic chip 100 realizes the expansion of the dynamic range, improves the detection sensitivity, and simultaneously realizes multiple detection lines on a single microfluidic chip, thus improving the experimental efficiency. In addition, compared with the conventional technology that requires multiple serial dilutions of samples to meet the concentration requirements of a microcavity with the single volume, the microfluidic chip 100 avoids multiple serial dilutions of the sample, thereby avoiding the waste of reagents and the risk of cross-contamination. Moreover, the above-mentioned arrangement of each microcavity of the microfluidic chip 100 can prevent mutual interference among different microcavities, which is beneficial to the effective identification of each microcavity by a fluorescence microscope.

As shown in FIG. 4 and FIG. 5, the microfluidic chip 100 may further comprises structures such as a substrate 104, an insulating layer 105, a defining layer 106, a hydrophilic layer 107, a conductive layer 108 , and a heating electrode 109. The substrate 104 may be a glass substrate. The insulating layer 105 is located on the substrate 104, in an example, the insulating layer 105 is a SiO₂ layer with a thickness of about 3000 Å. The defining layer 106 is located on the side of the insulating layer 105 away from the substrate 104, which defines each microcavity structure. For example, by patterning the defining layer 106, a plurality of grooves are formed in the defining layer 106, and the plurality of grooves constitute the plurality of microcavities of the microfluidic chip 100. In an example, the material of the defining layer 106 is photoresist. The hydrophilic layer 107 is located on the side of the defining layer 106 away from the substrate 104 and covers the bottom and sidewalls of the microcavity. The hydrophilic layer 107 has hydrophilic and oleophobic properties, and the hydrophilic performance of the interior of the microcavity (the sidewall and bottom of the microcavity) can be improved by setting the hydrophilic layer 107, which is beneficial to make the reaction system solution enter each microcavity more easily. In an example, the material of the hydrophilic layer 107 is SiO₂. The conductive layer 108 is located on the substrate 104 and is covered by the insulating layer 105, and the conductive layer 108 is electrically connected to the heating electrode 109. The conductive layer 108 is configured to apply an electrical signal (e.g., a voltage signal) to the heating electrode 109. After the heating electrode 109 receives the electrical signal, it can generate heat under the action of the electrical signal, thereby heating the microcavities to promote the dPCR reaction. In an example, the material of the heating electrode 109 is indium tin oxide (ITO).

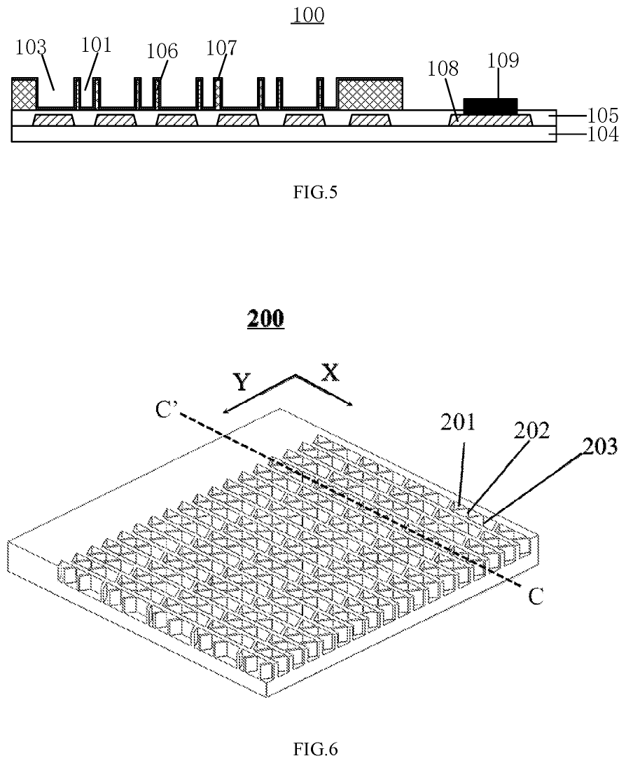

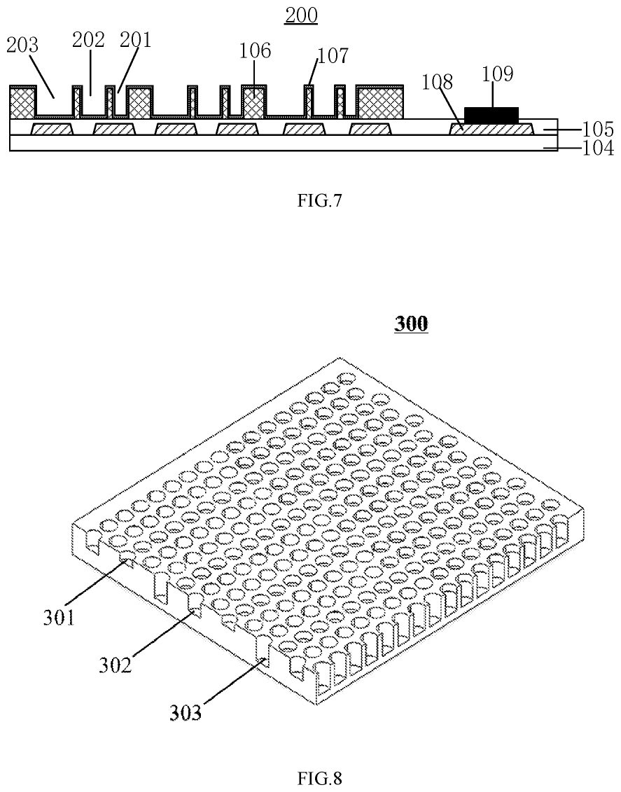

FIG. 6 illustrates a side view of the microfluidic chip 200, and FIG. 7 illustrates a cross-sectional view taken along line CC′ of FIG. 6. Referring to FIGS. 6 and 7, the microfluidic chip 200 has basically the same structure as the microfluidic chip 100 described in the above embodiments, except that the structure of the microcavity is different, that is, the microfluidic chip 200 also comprises structures such as the substrate 104, the insulating layer 105, the defining layer 106, the hydrophilic layer 107, the conductive layer 108, the heating electrode 109. In the following, for the sake of brevity, only the differences between the microfluidic chip 200 and the microfluidic chip 100 will be introduced, and the similarities will not be repeated.

As illustrated in FIG. 6 and FIG. 7, the microfluidic chip 200 comprises a plurality of microcavities comprising at least one first microcavity 201, at least one second microcavity 202 and at least one third microcavity 203. The volume of the first microcavity 201: the volume of the second microcavity 202: the volume of the third microcavity 203 is 1:2:3.

The first microcavity 201, the second microcavity 202 and the third microcavity 203 have the same depth. The depths of the first microcavity 201, the second microcavity 202 and the third microcavity 203 are all 30˜70 μm. In an example, the depths of the first microcavity 201, the second microcavity 202 and the third microcavity 203 are all 50 μm. The shape of the orthographic projection of the first microcavity 201 on the microfluidic chip 200 is an equilateral triangle, the shape of the orthographic projection of the second microcavity 202 on the microfluidic chip 200 is a parallelogram, and the shape of the orthographic projection of the third microcavity 203 on the microfluidic chip 200 is a trapezoid. Since the volume ratio of the first microcavity 201, the second microcavity 202 and the third microcavity 203 is 1:2:3 and they have the same depths, the area of the equilateral triangle: the area of the parallelogram: the area of the trapezoid is 1:2:3. The length of each of four sides of the parallelogram is equal to the length of a side of the equilateral triangle, the length of upper base of the trapezoid is equal to the length of the side of the equilateral triangle, and a length of a lower base of the trapezoid is twice the length of the side of the equilateral triangle. In an example, the length of the side of the equilateral triangle is 100 μm, the length of each of four sides of the parallelogram is 100 μm and the parallelogram consists of two equilateral triangles. The length of the upper base of the trapezoid is 100 μn and the length of the lower base is 200 μm, and the trapezoid consists of three equilateral triangles.

The first microcavities 201, the second microcavities 202 and the third microcavities 203 are all arranged in an array, the first direction X in FIG. 6 is the row direction, and the second direction Y is the column direction. As illustrated in the figure, viewed from left to right in each row, the microcavities are alternately arranged in a sequence of the first microcavity 201, the second microcavity 202 and the third microcavity 203. In each column, the microcavities in the same column have the same shape. Taking the first row in FIG. 6 as an example, the first four figures from left to right are an equilateral triangle, a parallelogram, a trapezoid, and an equilateral triangle. The first side of the parallelogram (that is, the left side of the parallelogram in the figure) is adjacent to and parallel to the first side of the equilateral triangle on its left (that is, the side of the equilateral triangle closest to the parallelogram). The second side of the parallelogram (that is, the right side of the parallelogram in the figure) is adjacent to and parallel to the first side of the trapezoid on its right side (that is, the left side of the trapezoid closest to the parallelogram). The second side of the trapezoid (that is, the right side of the trapezoid) is adjacent to and parallel to the second side of the equilateral triangle on its right side (that is, the side of the equilateral triangle closest to the trapezoid). The distance between the first side of the parallelogram and the first side of the left equilateral triangle is the first distance, the distance between the second side of the parallelogram and the first side of the trapezoid on the right is the second distance, the distance between the second side of the trapezoid and the second side of the equilateral triangle on the right is the third distance, the first distance, the second distance, and the third distance are equal. In an example, the first distance, the second distance, and the third distance are all 12.50 μm. In an example, the area of the orthographic projection of the plurality of microcavities of the microfluidic chip 200 on the microfluidic chip 200 accounts for 72.90% of the area of the microfluidic chip 200.

The microfluidic chip 200 has three types of microcavities with different volumes, namely a first microcavity 201, a second microcavity 202, and a third microcavity 203 with the same depth but different bottom areas, a ratio of volumes of the first microcavity 201, the second microcavity 202 and the third microcavity 203 is 1:2:3. The microfluidic chip 200 can allow the dilution concentration of the sample solution to have a wider selection range, without being restricted to only be diluted to a fixed concentration. For example, if a solution with a concentration of C1 is added to each microcavity, the concentration of C1 is sufficient for the third microcavity 203 to comprise one nucleic acid molecule, so the initial number of molecules of target nucleic acid molecules in the sample solution can be calculated mainly according to the measured value of the sample solution in the third microcavity 203. If a solution with a concentration of C2 (C2>C1) is added to each microcavity, the concentration of C2 may be too high for the third microcavity 203, making the number of nucleic acid molecules of the sample solution in each third microcavity 203 exceed the threshold requirement. However, the concentration of C2 may be sufficient for the second microcavity 202 to comprise one nucleic acid molecule, so the initial number of molecules of the target nucleic acid molecules in the sample solution can be calculated mainly according to the measured value of the sample solution in the second microcavity 202. If a solution with a concentration of C3 (C3>C2>C1) is added to each microcavity, the concentration of C3 may be too high for the third microcavity 203 and the second microcavity 202, making the number of nucleic acid molecules of the sample solution in each third microcavity 203 and each second microcavity 202 exceed the threshold requirement. However, the concentration of C3 may be sufficient for the first microcavity 201 to comprise one nucleic acid molecule, so the initial number of molecules of the target nucleic acid molecules in the sample solution can be calculated mainly according to the measured value of the sample solution in the first microcavity 201. The microcavity with the smaller volume is essentially equivalent to a greater dilution of the sample solution relative to the microcavity with the larger volume, so it can adapt to higher concentration solutions. In this way, it is possible to allow the sample solution to be diluted over a larger concentration range (for example, it can choose to dilute to concentration C1, C2 or C3), rather than having to be diluted to a fixed multiple as in the related art. Therefore, compared with the microfluidic chip in the related art, the microfluidic chip 200 realizes the expansion of the dynamic range, improves the detection sensitivity, and simultaneously realizes multiple detection lines on a single microfluidic chip, thus improving the experimental efficiency. In addition, compared with the conventional technology that requires multiple serial dilutions of samples to meet the concentration requirements of a microcavity with the single volume, the microfluidic chip 200 avoids multiple serial dilutions of the sample, thereby avoiding the waste of reagents and the risk of cross-contamination. Moreover, the above-mentioned arrangement of each microcavity of the microfluidic chip 200 can prevent mutual interference among different microcavities, which is beneficial to the effective identification of each microcavity by a fluorescence microscope.

FIG. 8 illustrates a side view of the microfluidic chip 300. Referring to FIG. 8, the microfluidic chip 300 has basically the same structure as the microfluidic chip 100 described in the above embodiments, except that the structure of the microcavity is different, that is, the microfluidic chip 300 also comprises structures such as the substrate 104, the insulating layer 105, the defining layer 106, the hydrophilic layer 107, the conductive layer 108 and the heating electrode 109. In the following, for the sake of brevity, only the differences between the microfluidic chip 300 and the microfluidic chip 100 will be introduced, and the similarities will not be repeated.

As illustrated in FIG. 8, the microfluidic chip 300 comprises a plurality of microcavities, and the plurality of microcavities 300 comprise at least one first microcavity 301, at least one second microcavity 302 and at least one third microcavity 303. The volume of the first microcavity 301: the volume of the second microcavity 302: the volume of the third microcavity 303 is equal to 1:2:4. The first microcavity 301, the second microcavity 302 and the third microcavity 303 are all cylindrical and have the same bottom area. In an example, the radius of the bottom of the first microcavity 301, the radius of the bottom of the second microcavity 302 and the radius of the bottom of the third microcavity 303 are 50 μm. Therefore, that is to say, the depths of the first microcavity 301, the second microcavity 302 and the third microcavity 303 are different, and a ratio of the depth of the first microcavity 301, the depth of the second microcavity 302 and the depth of the third microcavity 303 is 1:2:4. The depth of the first microcavity 301 is 25˜40 μm, the depth of the second microcavity 302 is 50˜80 μm, and the depth of the third microcavity 303 is 100˜160 μm. In an example, the depth of the first microcavity 301 is 25 μm, the depth of the second microcavity 302 is 50 μm, and the depth of the third microcavity 303 is 100 μm.

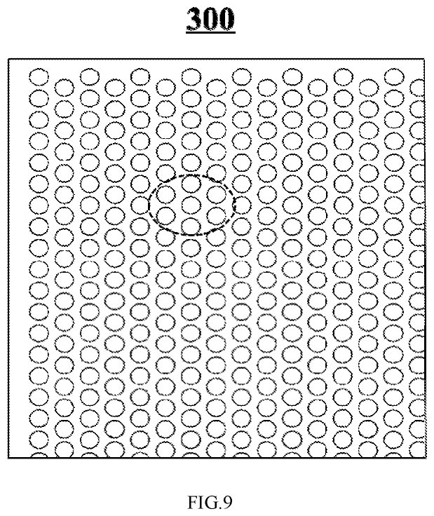

FIG. 9 illustrates an arrangement of a plurality of microcavities of the microfluidic chip 300 on the microfluidic chip 300. As illustrated in the figure, the plurality of microcavities are arranged on the microfluidic chip 300 in a manner of two-dimensional hexagonal dense arrangement, and the spacing between any two adjacent microcavities is 10˜80 μm. The area of the orthographic projection of the plurality of microcavities on the microfluidic chip 300 accounts for 24.67%-68.43% of the area of the microfluidic chip 300. The term “two-dimensional hexagonal dense arrangement” means that the plurality of microcavities are arranged in a honeycomb-like arrangement on the microfluidic chip 300 to maximize the utilization of the space area, but it is necessary to ensure that individual microcavities have an appropriate spacing to avoid mutual interference between the individual microcavities. As illustrated in the dashed box in FIG. 9, the two-dimensional hexagonal dense arrangement makes the line connecting the centers of the bottoms of the six adjacent microcavities form a regular hexagon, and another microcavity is arranged at the center of the regular hexagon, and the center of the bottom of the microcavity at the center coincides with the center of the regular hexagon. In an example, the plurality of microcavities of the microfluidic chip 300 are arranged in the manner of two-dimensional hexagonal dense arrangement, and the spacing between any two adjacent microcavities is 50 μm, and the area of the orthographic projection of the plurality of microcavities on the microfluidic chip 300 accounts for 40.18% of the area of the microfluidic chip 300. It should be noted that the “spacing” here does not refer to the distance between the centers of the bottoms of two adjacent microcavities, but refers to the distance between the sides closest to each other of two adjacent microcavities. Taking the seven microcavities (that is, seven circles) in the dotted box in FIG. 9 as an example, the distance between the tangent of the lowermost arc of the uppermost circle and the tangent of the uppermost arc of the circle located at the center of the regular hexagon is 50 μm.

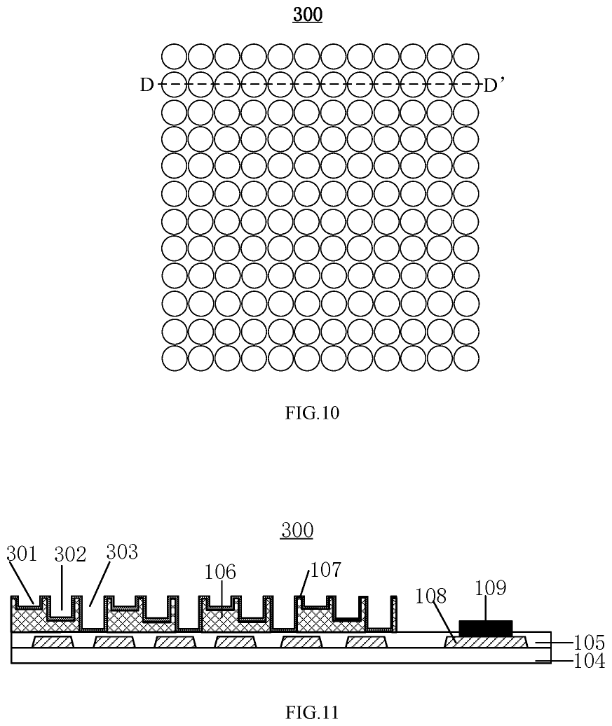

FIG. 10 illustrates another arrangement of the plurality of microcavities of the microfluidic chip 300 on the microfluidic chip 300, and FIG. 11 illustrates a cross-sectional view taken along the line DD′ of FIG. 10. As illustrates in the figures, the plurality of microcavities are arranged on the microfluidic chip 300 in the form of two-dimensional square lattice, and the spacing between any two adjacent microcavities is 10˜80 μm. The area of the orthographic projection of the plurality of microcavities on the microfluidic chip 300 accounts for 24.67%˜68.43% of the area of the microfluidic chip 300. It should be noted that the “spacing” here does not refer to the distance between the centers of the bottoms of two adjacent microcavities, but refers to the distance between the sides closest to each other of two adjacent microcavities. Taking the first two microcavities (that is, the first two circles) in the first column on the left in FIG. 10 as an example, the spacing between the tangent of the lowermost arc of the first circle and the tangent of the uppermost arc of the second circle is 10˜80 μm.

The term “two-dimensional square lattice” refers to that the plurality of microcavities are regularly arranged on the microfluidic chip 300, and the intersection of two adjacent rows of microcavities and two adjacent columns of microcavities is four microcavities. The lines connecting centers of the bottoms of the four microcavities form a square. This arrangement of the microcavities can maximize the utilization of the space area, but at the same time ensure that the individual microcavities have an appropriate spacing to avoid mutual interference between the individual microcavities. As illustrated in FIG. 10 and FIG. 11, along the direction of the DD′ line, the plurality of microcavities are alternately arranged in the sequence of the first microcavity 301, the second microcavity 302, and the third microcavity 303. In an example, the depth of the first microcavity 301 is 25 μm, the depth of the second microcavity 302 is 50 μm, and the depth of the third microcavity 303 is 100 μm.

It should be noted that although in FIGS. 8-10, the first microcavity 301, the second microcavity 302 and the third microcavity 303 are all illustrated as cylindrical, this is only an example, and the embodiments of the present disclosure do not limit the specific shapes of the first microcavity 301, the second microcavity 302 and the third microcavity 303. For example, the shape of the first microcavity 301, the second microcavity 302 and the third microcavity 303 comprises, but does not limit to, a cube, a quadrangular prism, a regular polyhedron, and the like.

The microfluidic chip 300 has three microcavities with different volumes, namely a first microcavity 301, a second microcavity 302 and a third microcavity 303 with the same bottom area but different depths. The ratio of volumes of the first microcavity 301, the second microcavity 302 and the third microcavity 303 is 1:2:4. The microfluidic chip 300 can allow the dilution concentration of the sample solution to have a wider selection range without being restricted to only be diluted to a fixed concentration. For example, if a solution with a concentration of C1 is added to each microcavity, the concentration of C1 is sufficient for the third microcavity 303 to comprise one nucleic acid molecule, so the initial number of molecules of target nucleic acid molecules in the sample solution can be calculated mainly according to the measured value of the sample solution in the third microcavity 303. If a solution with a concentration of C2 (C2>C1) is added to each microcavity, the concentration of C2 may be too high for the third microcavity 303, making the number of nucleic acid molecules of the sample solution in each third microcavity 303 exceed the threshold requirement. However, the concentration of C2 may be sufficient for the second microcavity 302 to comprise one nucleic acid molecule, so the initial number of molecules of the target nucleic acid molecules in the sample solution can be calculated mainly according to the measured value of the sample solution in the second microcavity 302. If a solution with a concentration of C3 (C3>C2>C1) is added to each microcavity, the concentration of C3 may be too high for the third microcavity 303 and the second microcavity 302, making the number of nucleic acid molecules of the sample solution in each third microcavity 303 and each second microcavity 302 exceed the threshold requirement. However, the concentration of C3 may be sufficient for the first microcavity 301 to comprise one nucleic acid molecule, so the initial number of molecules of the target nucleic acid molecules in the sample solution can be calculated mainly according to the measured value of the sample solution in the first microcavity 301. The microcavity with the smaller volume is essentially equivalent to a greater dilution of the sample solution relative to the microcavity with the larger volume, so it can adapt to higher concentration solutions. In this way, it is possible to allow the sample solution to be diluted over a larger concentration range (for example, it can choose to dilute to concentration C1, C2 or C3), rather than having to be diluted to a fixed multiple as in the related art. Therefore, compared with the microfluidic chip in the related art, the microfluidic chip 300 realizes the expansion of the dynamic range, improves the detection sensitivity, and simultaneously realizes multiple detection lines on a single microfluidic chip, thus improving the experimental efficiency. In addition, compared with the conventional technology that requires multiple serial dilutions of samples to meet the concentration requirements of a microcavity with the single volume, the microfluidic chip 300 avoids multiple serial dilutions of the sample, thereby avoiding the waste of reagents and the risk of cross-contamination. Moreover, the above-mentioned arrangement of each microcavity of the microfluidic chip 300 can prevent mutual interference among different microcavities, which is beneficial to the effective identification of each microcavity by a fluorescence microscope.

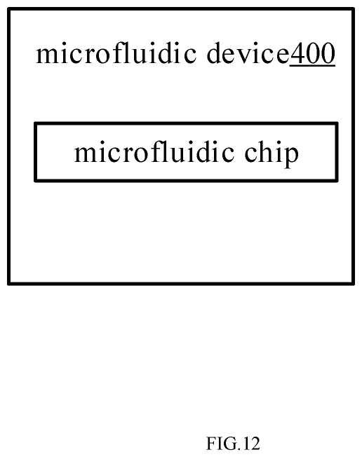

According to another aspect of the present disclosure, a microfluidic device 400 is provided, and FIG. 12 illustrates a block diagram of the microfluidic device 400. The microfluidic device 400 comprises the microfluidic chip described in any of the previous embodiments.

Since the microfluidic device 400 can have basically the same technical effect as the microfluidic chip described in the previous embodiments, for the sake of brevity, the technical effect of the microfluidic device 400 is not repeated here.

Yet another aspect of the present disclosure provides a method 500 for manufacturing a microfluidic chip. The method 500 can be applied to the microfluidic chip described in any of the previous embodiments. The method 500 is described below with reference to FIGS. 4 and 5.

Step 501: providing the substrate 104. The substrate 104 may be made of any suitable material. In an example, the substrate 104 is made of glass.

Step 502: forming a conductive layer on the substrate 104 at about 125° C. In an example, a molybdenum (Mo) film with a thickness of 200 Å, an aluminum (Al) layer with a thickness of 3000 Å, and a molybdenum (Mo) layer with a thickness of 800 Å are sequentially deposited on the substrate 104 to form a conductive film. The conductive film is patterned, such as exposure, development, etching, etc., to form the conductive layer 108

Step 503: depositing an insulating film on the conductive layer 108 at about 200° C., and patterning the insulating film to form the insulating layer 105 covering the conductive layer 108. In an example, the insulating layer 105 is a SiO₂ layer with a thickness of about 3000 Å.

Step 504: patterning the insulating layer 105 to form at least one via penetrating through the insulating layer 105 that exposes a portion of the conductive layer 108. In an example, the insulating layer 105 is etched in a dry etching machine to form vias, and the specific process is described as follows: etching for 10 s under the conditions of pressure of about 150 mtorr, power of about 800 W, and volume flow of O₂ of about 400 standard cubic centimeter per minute (sccm); etching for 200 s under the conditions of pressure of about 60 mtorr, power of about 800 W, and a ratio of gas volume flow of CF4 and O₂ about 200:50; etching for 30 s under the conditions of pressure of about 130 mtorr, power of about 800 w, and a ratio of gas volume flow of O₂ and CF₄ about 400:40; and etching for 160 s under the conditions of pressure of about 60 mtorr, power of about 800 w, and a ratio of gas volume flow of CF₄ and O₂ about 200:50.

Step 505: depositing a conductive film on the side of the insulating layer 105 away from the substrate 104, and then performing the steps of exposing, developing, etching, and striping to the conductive film to form the patterned heating electrode 109. In an example, the material of the heating electrode 109 is ITO. In an example, the heating electrode 109 may comprise multiple subsections that are separated from each other.

Step 506: depositing another insulating film on the side of the heating electrode 109 away from the substrate 104, and patterning the another insulating film to form another insulating layer (not illustrated in the figure) at least partially covering the heating electrode 109. In an example, the material of the another insulating layer is SiO₂. In another example, the another insulating layer comprises a layer of SiO₂ with a thickness of about 1000 Å and a layer of SiNₓ with a thickness of about 2000 Å, which are sequentially stacked.

Step 507: coating a shielding film on the side of the another insulating layer away from the substrate 104, and patterning the shielding film to form a shielding layer (not illustrated in the figure) defining openings. In an example, the specific steps of forming the shielding layer may comprise: coating a shielding film on the side of the another insulating layer away from the substrate 104, and then exposing, developing and etching the shielding film through a mask. Finally, post-curing the shielding film which is etched at 230° C. for about 30 minutes to form a shielding layer defining openings. The openings of the shielding layer may correspond to the position of the microcavities formed later. In an example, the material forming the shielding layer comprises chrome, chrome oxide, black resin.

Step 508: coating a defining film on the side of the shielding layer away from the substrate 104, and patterning the defining film to form a defining layer 106 defining a plurality of microcavities. In an example, the process of forming the defining layer 106 is described as follows: first, under the pressure of 30 Kpa, spin-coating the optical adhesive on the surface of the shielding layer away from the substrate 104 at a speed of 300 rpm for about 10 seconds, and then curing the optical adhesive at a temperature of 90° C. for 120 seconds. The above process is repeated twice to obtain a defining film. Next, the defining film is exposed through a mask, and then the exposed defining film is developed for 100 seconds with a developing solution, and then is etched. At a temperature of 230° C., the etched defining film is cured for 30 minutes, and finally a defining layer 106 defining a plurality of microcavities is obtained. The process steps used to form the multiple microcavities of the microfluidic chip 100, the multiple microcavities of the microfluidic chip 200, and the multiple microcavities of the microfluidic chip 300 are the same, except that masks of different shapes are used, so that different shapes of microcavities can be formed. The material of the defining layer 106 comprises photoresist.

Step 509: at 200° C., depositing an insulating film on the surface of the defining layer 106 away from the substrate 104, and exposing, developing and etching the insulating film to form a patterned layer. The patterned layer is treated with a 0.4% KOH solution for about 15 minutes to perform hydrophilic modification on the patterned layer, thereby forming a hydrophilic layer 107. The hydrophilic layer 107 covers the surface of the defining layer 106 away from the substrate 104 and covers the bottom and sidewall of each microcavity. In an example, the hydrophilic layer 107 is a SiO₂ layer with a thickness of about 3000 Å.

It should be noted that the manufacturing method may further comprise more steps, which may be determined according to actual requirements, which are not limited in the embodiments of the present disclosure. For the technical effect achieved by the manufacturing method, reference may be made to the description of the microfluidic chip above, which will not be repeated here.

In the description of the present disclosure, the orientations or positional relationships indicated by the terms “upper”, “lower”, “left”, “right”, etc. are based on the orientations or positional relationships shown in the accompanying drawings, and are only used for the convenience of describing the present disclosure. This disclosure is not required to be constructed and operated in the particular orientation and is therefore not to be construed as a limitation of the disclosure.

In the description of this specification, description with reference to the terms “an embodiment,” “another embodiment,” etc. means that a particular feature, structure, material, or characteristic described in connection with the embodiment is comprised in at least one embodiment of the present disclosure. In this specification, schematic representations of the above terms are not necessarily directed to the same embodiment or example. Furthermore, the particular features, structures, materials or characteristics described may be combined in any suitable manner in any one or more embodiments or examples. Furthermore, those skilled in the art may combine the different embodiments or examples described in this specification, as well as the features of different embodiments or examples, without conflicting each other. In addition, it should be noted that in this specification, the terms “first” and “second” are only used for description purposes, and cannot be understood as indicating or implying relative importance or implicitly indicating the number of indicated technical features.

As those skilled in the art will appreciate, although the various steps of the methods of the present disclosure are depicted in the figures in a particular order, this does not require or imply that the steps must be performed in that particular order, unless the context clearly dictates otherwise. Additionally or alternatively, multiple steps may be combined into one step for execution, and/or one step may be decomposed into multiple steps for execution. Furthermore, other method steps may be inserted between the steps. The inserted steps may represent improvements to the method such as those described herein, or may be unrelated to the method. Also, a given step may not be fully completed before the next step begins.

The above descriptions are merely specific embodiments of the present disclosure, but the protection scope of the present disclosure is not limited thereto. Any person skilled in the art can easily think of changes or substitutions within the technical scope disclosed by the present disclosure, which should be comprised within the protection scope of the present disclosure. Therefore, the protection scope of the present disclosure should be based on the protection scope of the claims.

1. A microfluidic chip comprising a plurality of microcavities,
wherein at least two of the plurality of microcavities have different volumes.

2. The microfluidic chip according to claim 1, wherein the plurality of microcavities comprise at least three types of microcavities with different volumes, and a ratio of volumes of the at least three types of microcavities with different volumes is 1:2˜4:3˜8.

3. The microfluidic chip according to claim 2, wherein the ratio of volumes of the at least three types of microcavities with different volumes is 1:4:8.

4. The microfluidic chip according to claim 3, wherein the plurality of microcavities comprise at least one first microcavity, at least one second microcavity and at least one third microcavity, the first microcavity, the second microcavity and the third microcavity have a same depth, a ratio of an area of a bottom of the first microcavity, an area of a bottom of the second microcavity, and an area of a bottom of the third microcavity is 1:4:8.

5. The microfluidic chip according to claim 4, wherein a shape of an orthographic projection of the bottom of the first microcavity, a shape of an orthographic projection of the bottom of the second microcavity and a shape of an orthographic projection of the bottom of the third microcavity on the microfluidic chip are circular.

6. The microfluidic chip according to claim 5, wherein a radius of the bottom of the first microcavity is 20 μm˜30 μm, a radius of the bottom of the second microcavity is 40 μm˜60 μm, and a radius of the bottom of the third microcavity is 56.57 μm ˜84.85 μm.

7. The microfluidic chip according to claim 4, wherein the depths of the first microcavity, the second microcavity and the third microcavity are 30 μm˜70 μm.

8. The microfluidic chip according to claim 4, wherein the first microcavities, the second microcavities and the third microcavities are arranged in an array, in a first direction, a row of second microcavities is arranged between two adjacent rows of third microcavities, and in a second direction, a column of second microcavities is arranged between two adjacent columns of third microcavities.

9. The microfluidic chip according to claim 8, wherein, a distance between centers of the bottoms of two adjacent first microcavities in the first direction is equal to a distance between the centers of the bottoms of two adjacent first microcavities in the second direction,
wherein a distance between the centers of the bottoms of two adjacent second microcavities in the first direction is equal to a distance between the centers of the bottoms of two adjacent second microcavities in the second direction, and
wherein a distance between the centers of the bottoms of two adjacent third microcavities in the first direction is equal to a distance between the centers of the bottoms of two adjacent third microcavities in the second direction.

10. The microfluidic chip according to claim 9,
wherein an intersection of the third microcavities in two adjacent rows and the third microcavities in two adjacent columns comprises four third microcavities, lines connecting centers of the bottoms of the four third microcavities form a square, one second microcavity is arranged at a center of the four third microcavities, and a center of the bottom of the second microcavity coincides with a midpoint of a diagonal of the square, and
wherein in the first direction or the second direction, one first microcavity is arranged between any two adjacent third microcavities, a center of the bottom of the first microcavity coincides with a midpoint of the line connecting centers of the bottoms of the two adjacent third microcavities, and in the first direction or the second direction, one first microcavity is arranged between any two adjacent second microcavities, a center of the bottom of the first microcavity coincides with a midpoint of the line connecting centers of the bottoms of the two adjacent second microcavities.

11. The microfluidic chip according to claim 1, wherein an area of an orthographic projection of the plurality of microcavities on the microfluidic chip accounts for 76.82% of an area of the microfluidic chip.

12. The microfluidic chip according to claim 2, wherein the ratio of volumes of the at least three types of microcavities with different volumes is 1:2:3.

13. The microfluidic chip according to claim 12,
wherein the plurality of microcavities comprise at least one first microcavity, at least one second microcavity and at least one third microcavity, the first microcavity, the second microcavity and the third microcavity have a same depth, and
wherein a shape of an orthographic projection of the first microcavity on the microfluidic chip is an equilateral triangle, a shape of an orthographic projection of the second microcavity on the microfluidic chip is a parallelogram, a shape of an orthographic projection of the third microcavity on the microfluidic chip is a trapezoid, and a ratio of an area of the equilateral triangle, an area of the parallelogram, and an area of the trapezoid is 1:2:3.

14. The microfluidic chip according to claim 13, wherein the first microcavities, the second microcavities and the third microcavities are arranged in an array, in a first direction, each row is alternately arranged in a sequence of the first microcavity, the second microcavity and the third microcavity, and in a second direction, the microcavities in a same column have a same shape.

15. The microfluidic chip according to claim 14,
wherein in each row, a first side of the parallelogram and a first side of the equilateral triangle adjacent to the parallelogram are parallel to each other, and a distance between the first side of the parallelogram and the first side of the equilateral triangle adjacent to the parallelogram is a first distance,
wherein a second side of the parallelogram and a first side of the trapezoid adjacent to the parallelogram are parallel to each other, and a distance between the second side of the parallelogram and the first side of the trapezoid adjacent to the parallelogram is a second distance,
wherein a second side of the adjacent trapezoid and a second side of the equilateral triangle adjacent to the adjacent trapezoid are parallel to each other, and a distance between the second side of the adjacent trapezoid and the second side of the equilateral triangle adjacent to the adjacent trapezoid is a third distance, and
wherein the first distance, the second distance and the third distance are the same.

16. The microfluidic chip according to claim 13,
wherein a length of each of four sides of the parallelogram is equal to a length of a side of the equilateral triangle, a length of an upper base of the trapezoid is equal to the length of the side of the equilateral triangle, a length of a lower base of the trapezoid is twice the length of the side of the equilateral triangle, the parallelogram consists of two equilateral triangles, and the trapezoid consists of three equilateral triangles.

17. The microfluidic chip according to claim 13, wherein the depths of the first microcavity, the second microcavity and the third microcavity are 30 μm˜70 μm.

18. The microfluidic chip according to claim 12, wherein an area of an orthographic projection of the plurality of microcavities on the microfluidic chip accounts for 72.90% of an area of the microfluidic chip.

19. The microfluidic chip according to claim 2, wherein the ratio of volumes of the at least three types of microcavities with different volumes is 1:2:4.

20. The microfluidic chip according to claim 19, wherein the plurality of microcavities comprise at least one first microcavity, at least one second microcavity and at least one third microcavity, an area of a bottom of the first microcavity, an area of a bottom of the second microcavity and an area of a bottom of the third microcavity are the same.

21. The microfluidic chip according to claim 20, wherein a depth of the first microcavity is 25 μm˜40 μm, a depth of the second microcavity is 50 μm ˜80 μm, and a depth of the third microcavity is 100 μm˜160 μm.

22. The microfluidic chip according to claim 20, wherein shapes of an orthographic projection of the bottom of the first microcavity, an orthographic projection of the bottom of the second microcavity and an orthographic projection of the bottom of the third microcavity on the microfluidic chip are circular, and a radius of the bottom of the first microcavity, a radius of the bottom of the second microcavity, and a radius of the bottom of the third microcavity are the same.

23. The microfluidic chip according to claim 19, wherein the plurality of microcavities are arranged in a manner of two-dimensional hexagonal dense arrangement or two-dimensional square lattice, and a spacing between any two adjacent microcavities of the plurality of microcavities is 10 μm˜80 μm.

24. The microfluidic chip according to claim 23, wherein an area of an orthographic projection of the plurality of microcavities on the microfluidic chip accounts for 24.67%˜68.43% of an area of the microfluidic chip.

25. The microfluidic chip according to claim 23, wherein the plurality of microcavities are arranged in the manner of two-dimensional hexagonal dense arrangement, and the spacing between any two adjacent microcavities of the plurality of microcavities is 50 μm, and an area of an orthographic projection of the plurality of microcavities on the microfluidic chip accounts for 40.18% of the area of the microfluidic chip.

26. A microfluidic device comprising the microfluidic chip according to claim 1.
